(19) United States
(12) Patent Application Publication
(10) Pub. No.: US 2006/0018836 A1
(43) Pub. Date: Jan. 26, 2006

(54) Method for treating human tumor cells with a newcastle disease virus strain having a p53 independent oncolytic effect

(75) Inventors: Laszlo K. Csatary, Alexandria, VA (US); Joseph Szeberenyi, Sziget (HU); Zsolt Fabian, Sziget (HU)

Correspondence Address: LAW OFFICES OF STUART J. FRIEDMAN, 28930 RIDGE ROAD, MT. AIRY, MD, 21771, US

(73) Assignee: United Cancer Research Institute, Alexandria, VA (US)

(21) Appl. No.: 10/997,154

(22) Filed: Nov. 24, 2004

Related U.S. Application Data
(60) Provisional application No. 60/524,725, filed on Nov. 25, 2003.

(51) Int. Cl. G01N 33/574; A61K 35/76

(52) U.S. Cl. 424/9.6; 435/7.23

(57) ABSTRACT

A method for treating p53-negative human tumor cells to induce apoptotic cell death thereof includes the step of infecting the tumor cells with the Herefordshire strain of Newcastle disease virus. The MOI is in the range of 100/1 to 1/200 cell/infected particle ratio.

### CROSS-REFERENCE TO RELATED APPLICATIONS

This is a non-provisional application based upon U.S. provisional application Ser. No. 60/524,725, filed Nov. 25, 2003, now pending.

### FIELD OF THE INVENTION

The present invention relates to a method for treating human tumor cells to induce apoptotic cell death thereof with a Newcastle Disease Virus (NDV) strain and, more particularly, to a method for treating human tumor cells with a strain having a p53 independent oncolytic effect.

### BACKGROUND OF THE INVENTION

Almost a century ago it was reported that patients suffering from gynecological cancers and vaccinated with Pasteur's rabies vaccine showed tumor regression, suggesting that vaccination can alter the progression of human cancers. Since then a group of almost 40 DNA and RNA viruses have been described which evidence the ability to selectively kill cancer cells. Among them can be found viruses of human diseases (such as smallpox, rabies, mumps) and viruses infecting birds. Since the idea of their therapeutic use in humans arose in the early 1960's, the oncolytic potential of some of these viruses has been confirmed in several human trials involving patients with cancers resistant to the traditional therapeutic modalities. Although these oncolytic viruses represent a promising possibility to find effective therapeutic strategies against resistant cancers, little is known about the mechanisms of their oncolytic cytotoxic effect.

Although the group of oncolytic viruses contains potentially dangerous human viruses (e.g., mumps virus) that are obviously inappropriate for human therapy, some of the others, including the avian paramyxovirus Newcastle Disease Virus (NDV), are not human pathogens. NDV was first described in the early 1900's as the contagious agent of the fatal avian disease known as chicken pest. It is a member of the paranyxoviridae family closely related to the infectious agent of human mumps. The structure of NDV is well characterized with particles containing a completely sequenced 15 kb long, single-stranded, non-segmented negative-sense RNA genome coding for six viral proteins. To date, many NDV forms have been described, however, these “field isolates” cannot be distinguished as distinct serotypes. Thus, their classification is based on their virulence (veloglenic, mesogenic or lentogenic forms) rather than on their serological differences. As an infectious agent, NDV causes serious infections in almost all birds, which, in the case of the velogenic forms, can lead to the death of the animals. Upon NDV infection, extensive apoptotic events can be detected in avian macrophages and lymphocytes of the periferial blood, although infection of the gastrointestinal and nervous systems is usually responsible for death. While NDV has a strong cytotoxic potential against different tumor cells, it is one of the few oncolytic viruses that naturally do not infect humans. No serious human infection was ever described, except mild conjunctivitis or tracheitis in people working with NDV vaccines. Although the molecular mechanism of the oncolytic action of NDV remains unclear at the present time, therapeutic trials have been performed in which different NDV isolates were found to be effective in some human tumors as diverse as hematological, gastrointestinal cancers and glioblastomas. NDV was also found to be cytotoxic for cultures of transformed avian and mammalian cells.

p53, a 53 kD nuclear phosphoprotein acts as a tumor suppressor protein by inhibiting cell proliferation in response to a variety of stress signals, including DNA damage. As a transcription factor, it regulates genes responsible for cell cycle arrest, repair of damaged DNA or induction of apoptosis. Since wild-type p53 has a very short half-life, its stabilization is crucial for its regulation. The ubiquitin ligase Mdm2 and lipid phosphatase PTEN are reported to be important regulatory proteins of p53. Mdm2 is a negative regulator of p53; upon Akt-mediated phosphorylation, Mdm2 stimulates ubiquitination and degradation of p53. In contrast, PTEN saves p53 from Mdm2-mediated degradation by inhibiting the Akt/Mdm2 pathway: as a lipid phosphatase, it eliminates the second messenger phosphatidylinositol-tris-phosphate (PIP₃) thereby preventing the activation of Akt protein kinase. Since the primary target for current chemo- and radiotherapies is the genomic DNA, the p53 status of the cancer cell has a fundamental effect on the outcome of anti-cancer treatments.

For this reason there is a pressing need for anti-cancer therapies which evidence p53-independent oncolytic action.

### SUMMARY OF THE INVENTION

It is, therefore, a primary object of the present invention to demonstrate the p53-independent oncolytic action of a purified, attenuated Herefordshire strain of Newcastle Disease Virus.

It is also an object of the present invention to demonstrate the effect of the Herefordshire strain on cell lines originating from human tumors.

The foregoing and other objects are achieved in accordance with the present invention by providing a method for treating p53 negative human tumor cells to induce apoptotic cell death thereof comprising the step of infecting the tumor cells with the Herefordshire strain of Newcastle Disease Virus.

In another aspect of the present invention, the infective virus titers (MOI: multiplicities of infection) are in the range of 100:1 to 1:200 of cells: infective Herefordshire strain particles.

According to the present invention, p53+human tumor cells were treated with the Herefordshire strain of Newcastle Disease Virus to demonstrate the cytotoxicity of the tumor cells to this strain and the practicality of a method for treating human tumor cells. The infection rate ratio varied from 100:1 to 1:200 of cell: infective Herefordshire strain particles. Particularly strong cytoxicity was noted at a cell:particle ratio of 1:10, although massive cell death was observed at much lower virus titers, i.e., 5:1 to 1:1. Human tumor cell lines were very sensitive to Herefordshire strain cytotoxicity, evidencing strong toxicity at cell: particle ratios as low as 100:1. It was also determined that Herefordshire strain induced cell death in p53-expressing and p53-depleted human tumor cells, such as human glioblastoma cells. Interstingly, the p53-depleted cells were observed to be 5 to 10 times more sensitive to the Herefordshire strain than other cell lines.

### BRIEF DESCRIPTION OF THE DRAWINGS

FIG. 1 is a graphical representation of the cytotoxicity of MTH-68/H on different cell lines.

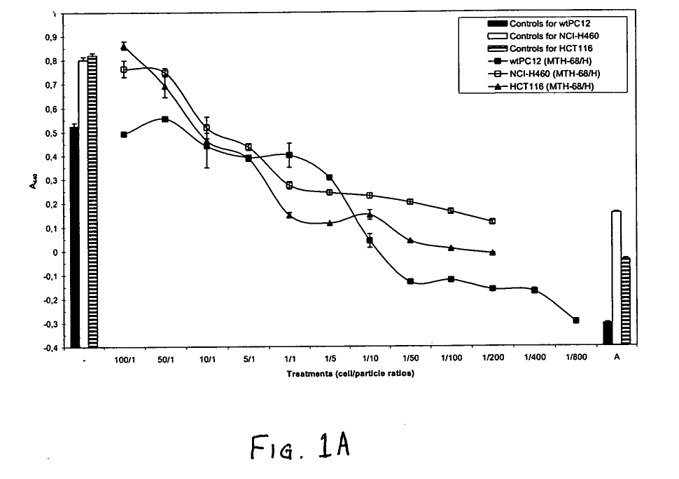

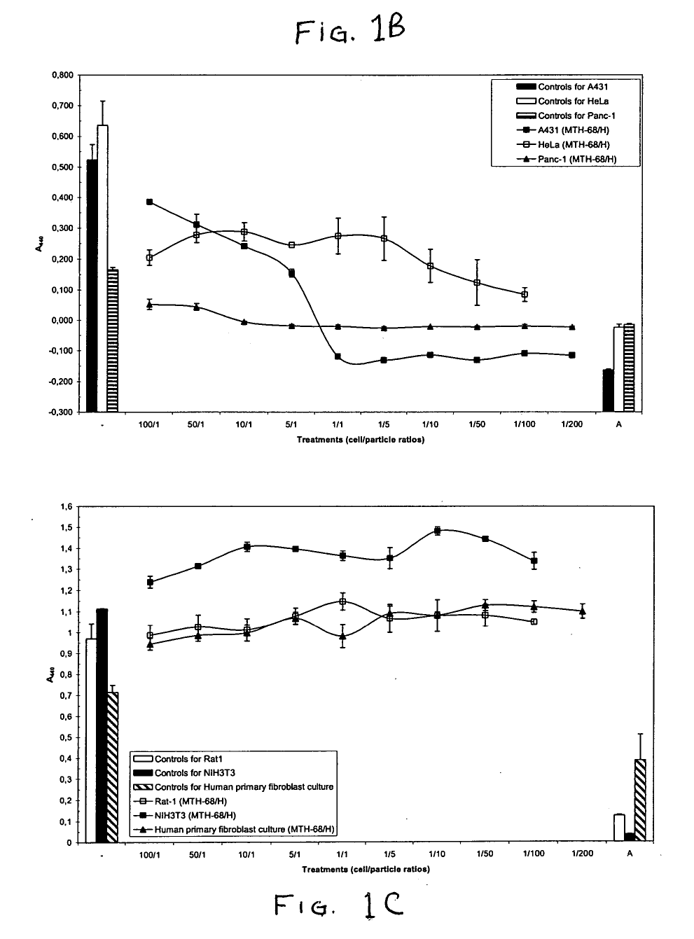

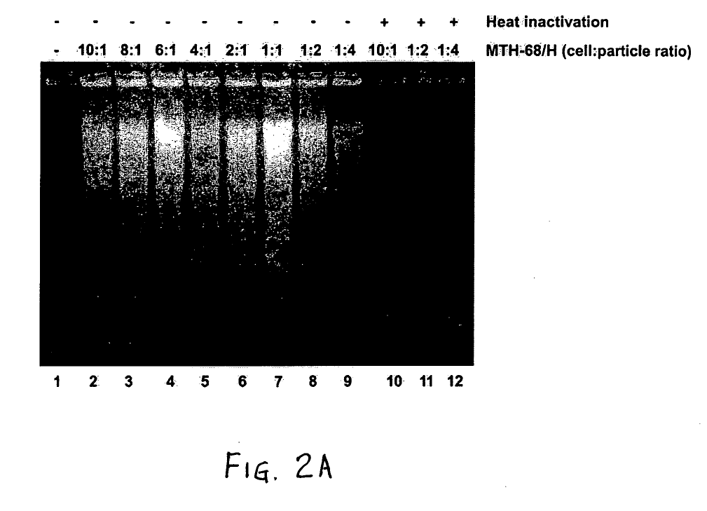

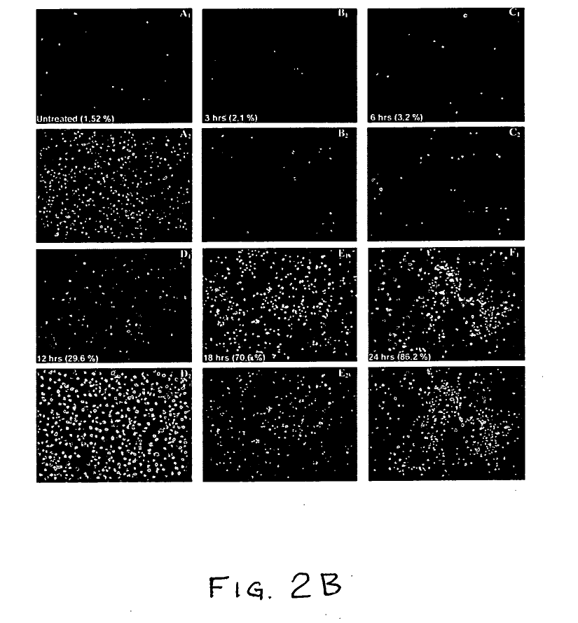

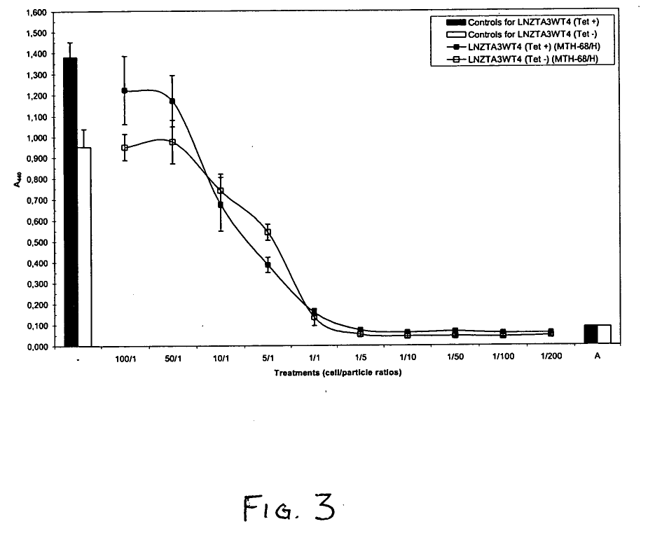

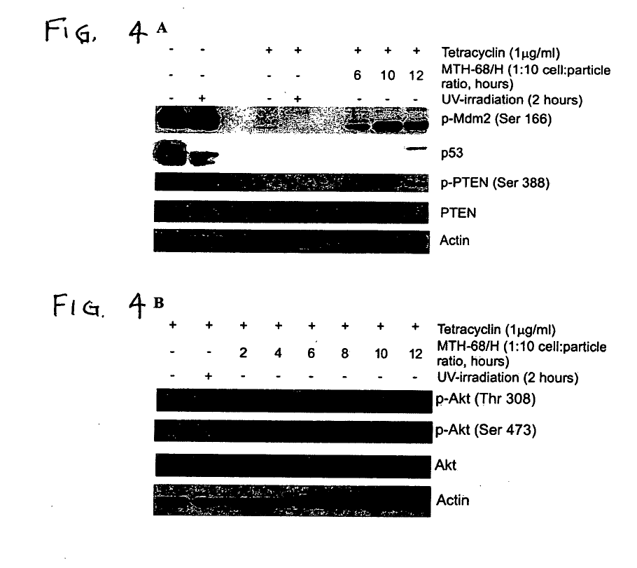

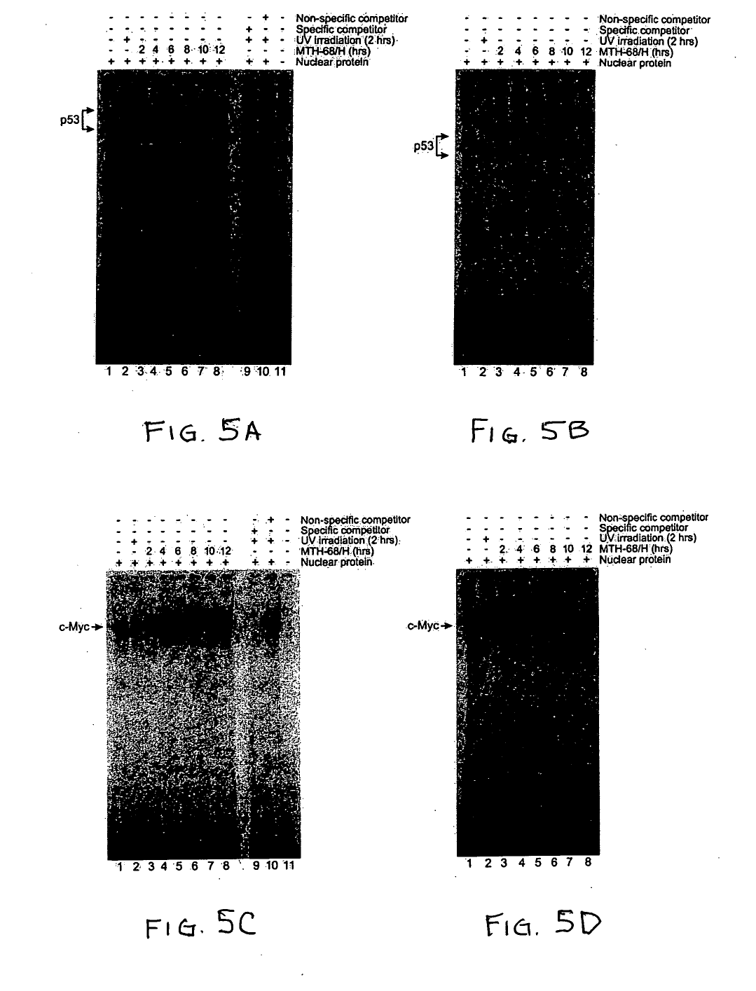

10⁴ (or in the case of the undifferentiated wild-type PC12 cells, 4×10⁴) cells were cultured in 24-well-format tissue culture plates. 24 hours after plating, cells were infected with the vaccine MTH-68/H, containing the Herefordshire strain, with different titers as indicated. For positive control, cells were treated with anisomycin (1 μg/ml); for negative control, they were grown in culture medium without treatment. After 72 hours of incubation, WST-1 assays were performed. No treatment and anisomycin controls are shown in the left (“−”) and right (“A”) sides of the FIGS. 1A-1C, respectively. FIG. 1A: p53 positive tumor cell lines; FIG. 1B: p53 negative tumor cell lines; FIG. 1C: non-transformed fibroblast cell lines.

FIG. 2 is an assay panel representation of apoptotic DNA fragmentation of HeLa cervical carcinoma cells treated with the vaccine MTH-68/H.

FIG. 2A: Electrophoretic analysis of internucleosomal DNA fragmentation. HeLa cells were infected with the vaccine MTH-68/H at multiplicities of infection indicated in the Figure (samples 1 to 9). In the same test, MTH-68/H particles were inactivated by boiling in culture medium for 30 minutes (samples 10-12). After 24 hours of infection DNA was extracted and examined by agarose gel electrophoresis as described herein.

FIG. 2B: Time kinetics of apoptosis in HeLa cells analyzed by TUNEL assay. TUNEL assay (panels A₁ to F₁) used to detect dying cells in HeLa cell cultures infected with the vaccine MTH-68/H for various durations was carried out as described herein. To visualize all the cell nuclei present in the culture, nuclei were counterstained with propidium-iodide (panels A₂ to F₂). The fraction of TUNEL positive cells is indicated in panels A₁ to F₁ Panels A₁ and A₂ show untreated, nearly confluent HeLa cell cultures. Panels C to F represent HeLa cultures treated with the vaccine MTH-68/H at 1:1 cell/particle ratio for the times indicated.

FIG. 3 is a graphical representation of the cytotoxic effect of MTH-68/H on p53-expressing and p53-depleted human glioblastoma cells.
LNZTA3WT4 cells were grown in the absence or presence of tetracyclin (1 μg/ml), infected with the vaccine MTH-68/H at multiplicities of infection indicated in the figure and WST-1 assays were performed.

FIG. 4 illustrates Western blot analysis of proteins of the p53 network in LNZTA3WT4 cells.
Cells were cultured either in the absence or presence of tetracyclin to induce or repress exogenous p53 transcription. Cultures were infected with the vaccine MTH-68/H as indicated in the Figure (cells UV-irradiated for 2 hours were used as control). To compare signals obtained with the individual antibodies, membranes were stripped between the primary antibody incubations and reprobed. Primary antibodies used in these tests are indicated on the right sides of the blots. Sample loading was controlled using anti-actin antibody, as indicated. Details of the Western blotting procedure are described herein. The panels FIGS. 4A and 4B show results of independent tests.

FIG. 5 illustrates panels showing DNA binding activity of p53 and c-Myc in MTH-68/H-infected cells.
WtPC12 (p53+, panels of FIGS. 5A and 5C), and LNZTA3WT4 (with repressed p53, panels of FIGS. 5B and 5D) cells were infected with the vaccine MTH-68/H using 1:10 cell:particle ratio for different times, as indicated. For controls, untreated (samples 1) or UV-irradiated cells (samples 2) were used. DNA binding reactions were performed using ³²P-labelled oligonucleotides carrying a p53 (panels of FIGS. 5A and 5B) or c-Myc binding sequence (panels of FIGS. 5C and 5D) as described herein. To prove the specificity of DNA binding, unlabelled competitor oligonucleotides containing specific (samples 9, p53 oligonucleotide for the panels of FIGS. 5A and 5B; c-Myc binding oligonucleotide for the panels of FIGS. 5C and 5D) or non-specific (samples 10, AP1 oligonucleotide) consensus sequences were used in excess amount. Samples 11 served as no-protein controls.

### DESCRIPTION OF THE PREFERRED EMBODIMENT

It has now been found, and evidence is presented herein in support of the findings, that an attenuated, non-pathogenic NDV strain, “H” (Herefordshire) strain, contained in the MTH-68/H vaccine, is directly oncolytic in several tumor cell lines of diverse tissue origin. The viral strain in MTH-68/H is able to replicate in transformed cell lines, but not in control cells and, significantly, the apoptosis inducing effect of the viral strain in MTH-68/H does not depend on the presence of functional p53 protein in the infected tumor cell lines.

It has been demonstrated that MTH-68/H destroyed a wide range of human cancer cells in culture (FIG. 1 and Table I), but was not cytotoxic for rat, mouse or primary human fibroblast cultures. This selective oncolytic effect was not affected by the tissue origin of the infected cell line: pancreas, glioblastoma, melanoma and cervical cancer cells were among the cell lines most susceptible to MTH-68/H-induced cell death. It was also found that the individual MTH-68/H sensitivity of tumor cell lines varied in a wide range (FIG. 1 and Table I). For example, the multiplicities of infection (MOI) required for 50% cell death of MCF-7 brest cancer and PANC-1 pancreas cancer cells were found to be 1:1 and 100:1 cell:particle ratios, respectively. Cancer cell lines, thus, have a common lesion that makes them susceptible to MTH-68/H-induced oncolysis, but the process is affected by the different genetic constitution of the different tumor cells. These variations in MTH-68/H susceptibility may be further increased by in vivo tumor characteristics. All of these conditions must be considered when virotherapy with the Herefordshire strain contained in MTH-68/H is planned.

A candidate for a gene/protein whose functional state may have a strong impact on the response of cells to MTH-68/H infection is p53. Its functional state is a main determinant of the response of tumors to chemo- and radiotherapy. It was found the apoptosis-inducing effect of MTH-68/H was not influenced by the p53 status of the cell line in human tumor cell lines of diverse p53 status. Most importantly, a human glioblastoma cell line with controllable p53 expression displayed no difference toward MTH-68/H-induced cytotoxicity in its p53- and p53+states (FIGS. 3 and 4). These observations may have very important clinical implications since progression of human tumors often leads to the loss of p53 function. Accordingly, Herefordshire strain therapy, for example with the MTH-68/H vaccine, can be a promising alternative treatment in patients with p53-negative, highly chemoresistant malignancies.

To demonstrate the p 53 independent cytotoxicity of the H (Herefordshire) strain, several studies were conducted on various human cell lines. The viral vaccine known as MTH-68/H, developed by United Cancer Research Institute (Ft. Lauderdale, Fla.) and available from UCRI Hungary Ltd. of Budapest, Hungary, contains highly purified, attenuated, mesogenic H (Herefordshire) Newcastle Disease virus strain (hereinafter “Herefordshire strain”) and was used as the source of the Herefordshire strain. Cell death caused by this strain of Newcastle Disease Virus comes in the form of apoptosis. As used hereinbefore and hereinafter, the vaccine designation “MTH-68/H” refers to the aforementioned viral vaccine containing highly purified, attenuated Herefordshire strain.

Cell Lines

The main features of the cell lines used in these studies are summarized in Table I. The p53 status of each cell line and its relative sensitivity to the Herefordshire strain is presented in Table I. PC12 cells were cultured in Dulbecco's modified Eagle medium with 4.5 g/L glucose (DMEM) supplemented with 10% horse and 5% fetal bovine serum (FBS). HeLa, U373 and MCF-7 cells were cultured in DMEM containing 10% FBS; NIH3T3 and Rat-1 fibroblasts in DMEM supplemented with 10% calf serum. Primary human fibroblasts were maintained in DMEM containing 20% FBS. PANC-1 cells were grown in PRMI 1640 with phenol red supplemented with MEM-non essential amino acid solution and 10% FBS, 2 mM L-glutamine and 10% MEM-sodium

TABLE I
Main characteristics of the cell lines

| | | | Relative |
|---|---|---|---|
| | | | MTH-68/H |
| Cell line | Tissue origin | p53 status | sensitivity³ |
| Primary fibroblast | Human | NA¹ | None |
| NIH3T3 | Mouse embryonic fibroblast | NA | None |
| Rat-1 | Rat embryonic fibroblast | NA | None |
| HT-25 | Human colon carcinoma | NA | +++ |
| HT-29 | Human colorectal | CGT/CAT mutation in codon 273 (40) | ++++ |
| | adenocarcinoma | | |
| HCT-116 | Human colon carcinoma | wtp53+ (58) | +++ |
| DU-145 | Brain metastasis of human | Both alleles are mutated: Pro²²³Leu and Val²⁷⁴Phe (59) | +++ |
| | prostate adenocarcinoma | | |
| PC-3 | Bone metastasis of human | One allele is deleted; | ++++ |
| | prostate adenocarcinoma | Point mutation in codon 138 resulting frameshift leading to the early | |
| | | termination (59) | |
| PANC-1 | Ductal epithelioid carcinoma | CGT/CAT mutation in codon 273 (60) | +++++ |
| | of human pancreas | | |
| MCF-7 | Human breast | wtp53+ (61) | ++ |
| | adenocarcinoma | | |
| HeLa | Human cervix | HPV16 E6+; | +++++ |
| | adenocarcinoma | low p53 expression (42) | |
| NCI-H460 | Pleural metastasis of human | Elevated p53 mRNA expression.² | +++ |
| | large cell type lung carcinoma | | |
| U373 | Human astrocytoma | CGT/CAT mutation in codon 273 (62) | +++ |
| LNZTA3WT4 | Human glioblastoma | Endogenous p53 is inactivated. The cell line is stably transfected by | ++++ |
| | | wtp53 cDNA driven by CMV promoter and repressed by a tetracyclin | |
| | | repressor.² | |
| A431 | Human epidermoid | CGT/CAT mutation in codon 273 (41) | ++++ |
| | carcinoma | | |
| HT-168-M1/9 | Human melanoma | NA | ++++ |
| HT199 | Human melanoma | NA | ++++ |
| WM983B | Human melanoma | NA | ++++ |

| Relative | |
|---|---|
| MTH-68/H | Cell:particle ratio causing |
| sensitivity | 50% cytotoxicity |
| +++++ | 100:1-10:1 |
| ++++ | 10:1-5:1 |
| +++ | 5:1-1:1 |
| ++ | 1:1-1:10 |
| + | 1:10-1:200 |
| No | Lower than 1:200 |

¹NA: No data available;
²Based on the description of the American Tissue Culture Collection.
³Assessment of the relative MTH-68/H sensitivity was based on MTH-68/H titers sufficient to induce 50% cytotoxicity as follows:

pyruvate. HCT-116, HT-25, HT-168-M1/9, HT-199, WM983B cells were grown in RPMI 1640 containing 5% FBS and 3 mM L-glutamine. DU-145, PC-3, HT-29 and NC1-H460 cells were cultured in DMEM-Ham's F12 (1:1) with 10% FBS. A431 cells were grown in DMEM-Ham's F12 (1:1) with 5% FBS. The human glioblastoma cell line LNZTA3WT4 was cultured in DMEM containing 10% FBS, 2 mM L-glutamine and 1 μg/ml tetracyclin. LNZTA3WT4 was derived from the LN-Z308 cell line. The parental LN-Z308 cells do not express endogenous p53 due to an internal rearrangement of the endogenous p53 gene. Subsequent transfections of the p53⁻ parental cells with a plasmid containing wild-type p53 (wtp53) cDNA resulted in stable transfectants like the LNZTA3WT4 clone with tetracyclin-regulated expression of wtp53 under the control of a CMV promoter and a tetracyclin repressor: wtp53 is expressed only in the absence of the antibiotic.

### Detection of Cytotoxicity Using the WST-1 Cell Proliferation Assay

Proliferation and viability of cell lines were analyzed using the WST-1 kit of Roche Molecular Biochemicals. This assay measures metabolically active mitochondria in cultured cells. Cells were grown in tissue culture grade, 24-well plates, in 1 ml culture medium as described above and infected with MTH-68/H for 72 hours. For positive apoptosis control, cells were treated for 24 hours with 1 μg/ml anisomycin, whereas for negative controls cells were treated with vehicle. For the WST-1 cell proliferation assay, treated cells were incubated for various times (from 90 to 240 minutes depending on the cell type) in culture medium containing 0.1 volume of WST-1 reagent. At the end of the treatment period 100 μl samples were transferred to a 96-well plate. The absorbance of the formazan formed was measured by a multiwell spectrophotometer at 440 nm. Measurements were performed in triplicates.

Apoptosis Assays

Electrophoretic Detection of Internucleosomal

Fragmentation of Chromosomal DNA

5×10⁶ cells were cultured in DMEM (Dulbecco's modified Eagle medium) containing sera for 24 hours. Treatments were carried out as indicated in the descriptions of each of the Figures. Following incubation, cells were collected by scraping them into their own medium and then centrifuged at 600×g for 3 minutes. The cells were solubilized on ice in TE buffer (pH 7.4) containing 2% SDS. After centrifugation at 13500 rpm for 20 minutes at 4° C., soluble DNA in the supernatant was extracted with phenol/chloroform, and precipitated with ethanol. The dried precipitates were dissolved in TE buffer and treated with DNase free RNase A at 37° C. for at least 1 hour. DNA fragments were separated by electrophoresis in 1.8% agarose gels, and visualized on a UV transilluminator after staining with SYBR Gold (Molecular Probes, Eugene, Oreg.).

TUNEL Assay

For all treatments, 10⁵ cells were seeded in 8-well chamber slides, cultured for 24 hours and treated with MTH-68/H. The cells were fixed in 0.14 M phosphate-buffered saline (pH 7.4, PBS) containing 4% paraformaldehyde and 2.5% DMSO at 4° C. for 60 minutes, washed in PBS three times for 5 minutes and permeabilized in PBS containing 0.1% Triton X-100, 0.1% sodium citrate at 4° C. for 2 minutes. Cells were washed and stained using FITC-labeled dUTP and terminal deoxynucleotide transferase at 37° C. for 60 minutes. TUNEL reaction was terminated by 2×SSC (0.3 M NaCl/0.03 M Na-citrate) for 10 minutes and cells were counterstained with propidium-iodide/RnaseA solution for 10 minutes at room temperature. Samples were washed with distilled water and covered using Vectashield H-1000 mounting solution (Vector, Burlingame, Calif.).

Western Blotting

Immunoblot analysis using antibodies against proteins indicated in the Figures was performed according to the manufacturer's recommendations. Cells were collected by scraping and centrifuged at 600×g for 3 minutes, lysed in ice-cold lysis buffer containing 50 mM Tris (pH 7.4), 150 mM NaCl, 10% glycerol, 1 mM EGTA, 1 mM Na-orthovanadate, 5 μM ZnCl₂, 100 mM NaF and 1% Triton X-100. The lysis buffer was supplemented with 10 μl/ml phosphatase inhibitor cocktail 1 and 1 tablet/10 ml protease inhibitors (Complete, Mini EDTA-free tablets, Roche Hungary). Following lysis for 10 minutes on ice, samples were centrifuged at 13500 rpm for 10 minutes at 4° C. Protein concentration was determined using the Bio-Rad Protein RCDC assay system (Bio-Rad Hungary, Budapest). Thirty μg protein for each sample was resolved by SDS polacrylamide gel electrophoresis in 10% gels. The proteins were transferred to PVDF membranes (Amersham Pharmacia Biotech AB., Uppsala, Sweden), treated with appropriate antibodies and immune complexes were visualized using an Enhanced Chemiluminescence Detection kit (Amersham Pharmacia Biotech AB) following the manufacturer's instructions. The following antibodies were used: phosphor-Mdm2 (pAb), p53 (pAb), phosphor-PTEN (Ser388, pAb), PTEN (pAb), phosphor-Akt (Thr308 pAb), phosphor-Akt (Ser473 pAb), Akt (pAb) antibodies from Cell Signaling (Beverly, Mass.) and Actin (mAb, AB-1) antibody from Oncogene (Merck Ltd., Budapest, Hungary).

Electrophoretic Mobility Shift Assay (EMSA)

Nuclear extracts were prepared as described by Xu & Cooper in “Identification of a candidate c-mos repressor that restricts transcription of germ cell-specific genes”; Mol Cell Biol 1995; 15: 5369-5375. All subsequent steps were performed at 4° C. Cell pellets were washed twice in ice cold phosphate-buffered saline (PBS) and resuspended in 10 volumes of buffer containing 10 mM HEPES pH 7.9, 1.5 mM MgCl₂, 10 mM KCl, 0.5 mM dithiothreitol (DTT), protease inhibitors (Complete, Mini EDTA-free tablets, Roche, Hungary), phosphatase inhibitors (Phosphatase Inhibitor Cocktail I, Sigma) and placed on ice for 10 minutes. Nuclei were collected by centrifugation and resuspended in 2 volumes of 20 mM HEPES (pH 7.9), 25% glycerol, 420 mM NaCl, 1.5 mM MgCl₂, 0.2 mM EDTA, 0.5 mM DTT, protease inhibitors and phosphatase inhibitor cocktail I, and placed on ice for 20 minutes. After centrifugation, protein concentrations of the supernatants were determined with the Bio-Rad RCDC Protein Assay Kit. 5′-end labeling of the p53 oligonucleotide was performed using [γ-³²P]-ATP (institute of Isotopes Co., Ltd., Budapest, Hungary) and T4 polynucleotide kinase (Amersham Pharmacia Biotech Inc.) according to the manufacturer's protocol. Double-stranded p53 oligonucleotide containing the consensus binding site for p53 (5′-TACAGAACATGTCTAAGCATGCTGGGGACT-3′) was obtained from Santa Cruz Biotechnology Inc., (Santa Cruz, Calif.) and double-stranded oligonucleotide containing the consensus binding site for c-Myc (5′-TGTGCGGCCACGTGTCGCGAGGCCCGG-3′) was obtained from Amitof (Boston, Mass.). Ten μg of nuclear proteins were mixed with 100 ng nonspecific single-stranded oligonucleotide in 411 buffer containing 10 mM HEPES (pH 7.5), 1 mM EDTA, 100 mM NaCl, 2 μg poly(dI-dC) for c-Myc or 3 μg poly(dI-dC) for p53 binding reactions and the final reaction volume was adjusted to 18 μl with distilled water. After a 15-minute pre-incubation at room termperature, 10⁵ cpm of ³²P-labelled oligonucleotide was added and the incubation at room temperature was continued for another 30 minutes. DNA-protein complexes were electrophoresed in 5% non-denaturing polyacrylamide gels using a TRIS-Base, borate, EDTA buffer (pH 8.3) for 2.5 hours at 200 V. The gel was dried and analyzed by a Cyclone PhosphorImager (Packard Instrument Co. Inc., Meriden, Conn.).

Quantification of Infective MTH-68/H Particles

6×10⁸ primary chicken embryonic cells were infected with 0.5 ml of the supernatants of cancer cells treated with different MOI of MTH-68/H for 72 hours. After 3 days of incubation, intracellular viruses were released by sonic treatment and titrated by plaque assay.

With reference to FIGS. 1-5 and Tables I and II, there can be seen the results obtained by infecting various tumor cell lines with the Herefordshire strain utilized in the form of the MTH-68/H vaccine.

Selective Sensitivity of Cancer Cell Lines to MTH-68/H

Several, mostly human tumor cell lines with wild-type (FIG. 1A) or mutated p53 genes (FIG. 1B) as well as non-transformed fibroblast cell lines (FIG. 1C) were tested for MTH-68/H cyutotoxicity (see Table I). Using the WST-1 proliferation assay, MTH-68/H was tested in a wide range of multiplicity of infection (MOI; from 100:1 to at least 1:200 cell:infective particle ratios). WST-1 assay experiments with wtPC12 cells confirmed that the cytotoxic effect of MTH-68/H is dose dependent in this cell line. MTH-68/H exerted strong cytotoxicity at a cell:particle ratio of 1:10. It is noteworthy that DNA fragmentation assays or microscopic observations (not shown) revealed massive cell death even at much lower virus titers (5:1 to 1:1 cell:particle ratios). These observations suggest that some of the dying cells might have relatively intact mitochondrial functions and, therefore, the sensitivity of apoptosis assays may exceed that of the WST-1 test. Other p53+human tumor cell lines (like the lung cancer cell line NCI-H460 and the colon carcinoma cell line HCT-116, see FIG. 1A) were also sensitive to MTH-68/H induced cytotoxicity.

Tumor cell lines with impaired p53 function were also found sensitive to MTH-68/H cytotoxicity (FIG. 1B and Table I). In two of them (HeLa cervical cancer cells and PANC-1 pancreas carcinoma cells) even the lowest MTH-68/H titer tested (100:1 cell:particle ratio) was strongly toxic. Other cell lines with reduced p53 function (e.g., HT-29 colorectal adenocarcinoma, DU-145 and PC-3 metastatic cancer cells with prostate origin, etc.) were also sensitive to MTH-68/H infection, although quantitative variations among tumor cell lines were apparent (Table I).

It should be noted that all the human tumor cell lines tested were found to be sensitive to MTH-68/H (Table I). In contrast, non-transformed fibroblasts (NIH3T3 and Rat-1 rodent fibroblast cell lines and human primary fibroblast cultures) were found to be highly resistant to MTH-68/H cytotoxicity. They continued their proliferation even at slightly higher rates than untreated control cells, while retaining their ability to carry out apoptotic cell death in response to anisomycin treatment (FIG. 1C).

Replication of MTH-68/H in HeLa and MCF-7 Cells

As described hereinbefore, MTH-68/H triggered cell death in this in vitro system in the absence of immune cells, suggesting that immunological processes are not required for tumor cell killing. To demonstrate that active viral replication-mediated oncolysis takes place in cultured tumor cells, HeLa and MCF-7 cell cultures were treated with MTH-68/H (100:1 cell:virion ratio), and the production of infectious viruses in the medium was evaluated. After 24 hours of infection, the culture media were collected and transferred to fresh, non-infected cull cultures, incubated for an additional 24 hours and analyzed by phase-contrast microscopy. The results of this test indicated that the “preconditioned” media induced cell death in both HeLa and MCF-7 cells, suggesting the presence of a cytotoxic agent in the medium of MTH-68/H infected tumor cell cultures.

To demonstrate that MTH-68/H virions replicate successfully in transformed cells, supernatants of PC12, HeLa, NIH-3T3 and Rat-1 cell cultures infected for 72 hours with the virus were re-titrated to determine the number of infectious particles (Table II). At very low multiplicities of infection (100:1, 50:1 cell:particle ratios), virus titers were increased both in PC 12 and HeLa cel cultures during the 72 hour infection period, clearly indicating that virus replication takes place in these cultures. In cultures infected with higher MTH-68/H titers, the number of the particles did not change significantly or declined, most likely as a consequence of massive cell death at higher virus titers limiting the capacity of cultures to produce new virions. In contrast, no increase of the number of infective particles was detected in NIH-2T3 and Rat-1 fibroblasts (Table II). The low number of particles found in cultures at the end of incubation infected with high initial virus titers (1:5 or higher cell:particle ratio) probably represent surviving virions rather than newly replicated particles. All these observations and considerations suggest that MTH-68/H actively replicates in PC12 and HeLa cells, but not in non-transformed fibroblasts.

Apoptotic Cell Death in HeLa Human Cervical Cancer Cells Upon MTH-68/H Infection

Electrophorectic analysis of DNA fragmentation revealed that MTH-68/H induced strong internucleosomal chromatin cleavage—a hallmark of apoptosis—in HeLa cells, even at low virus titers (FIG. 2A). A 30 minute heat inactivation of MTH-68/H completely abolished virus-induced apoptosis, indicating that live virions are required to kill HeLa cells (FIG. 2A, lanes 10 to 12). Furthermore, HeLa cells proved to be much more sensitive to MTH-68/H infection than wild-type PC 12 cells in the DNA fragmentation assays: 10:1 cell:particle ratio was sufficient to cause apoptotic DNA ladders (FIG. 2A), while the cell:particle ratio with the same effect in PC12 cells was 1:1 to 2:1. Moreover, 100:1 cell:particle ratio used in the WST-1 assay (FIG. 2B) was also sufficient to cause full-blown DNA fragmentation in HeLa cells (not shown).

Quantitative analysis of MTH-68/H-induced apoptosis of HeLa cells by TUNEL assay confirmed the results of electrophoretic analysis of DNA fragmentation (FIG. 2B). HeLa cells were infected with MTH-68/H (1:1 cell:particle ratio). Fragmented DNA was end-labeled by FITC-dUTP to identify the apoptotic cells. To determine the total cell number, cells were counterstained with propidium-iodide. The fraction of apoptotic cells was determined at different time points after infection. TUNE: positivity of HeLa cultures started to increase from the basal level of 1-3% (FIG. 2B, panels A to C) after 12 hours of treatment with MTh-68/H (almost 30% in panel D), and

TABLE II

| | wtPC-12 | | HeLa | | NIH3T3 | | Rat-1 | |
|---|---|---|---|---|---|---|---|---|
| | Virus titers (MTH-68/H particles/ml) | | | | | | | |
| Cell type | At the | | At the | | At the | | At the | |
| Treatment | beginning of | After 72 | beginning of | | beginning of | After 72 | beginning of | After 72 |
| (cell:particle ratio) | incubation | hrs | incubation | After 72 hrs | incubation | hrs | incubation | hrs |
| Untreated | — | <10¹ | — | <10¹ | — | <10¹ | — | <10¹ |
| Anisomycin (1 μg/ml) | — | <10¹ | — | <10¹ | — | <10¹ | — | <10¹ |
| 100:1 | 4 × 10² | 4 × 10³ | 10² | 6.3 × 10⁴ | 10² | <10¹ | 10² | <10¹ |
| 50:1 | 8 × 10² | 4 × 10³ | 2 × 10² | 1.5 × 10⁴ | 2 × 10² | <10¹ | 2 × 10² | <10¹ |
| 10:1 | 4 × 10³ | 4 × 10³ | 10³ | 1.5 × 10⁴ | 10³ | <10¹ | 10³ | <10¹ |
| 5:1 | 8 × 10³ | 4 × 10³ | 2 × 10³ | 2.5 × 10⁴ | 2 × 10³ | <10¹ | 2 × 10³ | <10¹ |
| 1:1 | 4 × 10⁴ | 2.5 × 10⁴ | 10⁴ | 1.5 × 10⁴ | 10⁴ | <10¹ | 10⁴ | <10¹ |
| 1:5 | 2 × 10⁵ | 2.5 × 10⁴ | 5 × 10⁴ | 1.5 × 10⁴ | 5 × 10⁴ | 1.3 × 10² | 5 × 10⁴ | 6 × 10¹ |
| 1:10 | 4 × 10⁵ | 4 × 10⁴ | 10⁵ | 10⁵ | 10⁵ | 2.1 × 10² | 10⁵ | 8 × 10¹ |
| 1:50 | 2 × 10⁶ | 1.5 × 10⁴ | 5 × 10⁵ | 10⁵ | 5 × 10⁵ | 1.4 × 10³ | 5 × 10⁶ | 7.5 × 10² |
| 1:100 | 4 × 10⁸ | 1.5 × 10⁵ | 10⁶ | 1.5 × 10⁵ | 10⁶ | 1.9 × 10³ | 10⁶ | 7.2 × 10² |

increased rapidly afterwards (70% and 86% at 18 and 24 hourse of treatment, respectively, FIG. 2B, panels E and F). Similar time course was observed with other human carcinoma cell lines (e.g., HT-29, LNZTA3WT4, MCF-7, data not shown), suggesting that apoptotic processes appear to be responsible for the cellular cytotoxic effect of MTH-68/H observed in the WST-1 assays.

MTH-68/H Induces Cell Death in p53-Expressing and p53-Depleted Human Glioblastoma Cells

MTH-68/H induces cell death in cell lines expressing wild-type p53 (e.g., wtPC12, HCT116 or MCF-7 cells) or mutated forms of p53 (e.g., DU-145, HT-29 or A431) or reduced levels of p53 (HeLa or PC-3). These cell lines, however, have very different genetic background. To further analyze the role of p53 protein in MTH-68/H-induced apoptosis, use was made of a cell line with inducible/repressible p53 expression.

The human glioblastoma cell line LNZTA3WT4 has two useful features important in studies with MTH-68/H. First, due to genomic rearrangements, it does not have endogenous wild-type p53 expression, but produces p53 protein encoded by a transfected cDNA under the control of a tetracyclin-repressible promoter: p53 is expressed in the absence of tetracyclin in the medium, but not in its presence. Second, glioblastoma belongs to the group of human malignancies that responded promisingly to MTH-68/H treatment in previous clinical trials. LNZTA3WT4 cells were infected with MTH-68/H in the presence or absence of tetracyclin and analyzed by the WST-1 assay (FIG. 3): the cells displayed very similar curves of MTH-68/H cytotoxicity both in the absence and presence of tetracyclin. Moreover, the p53-depleted cells were 5- to 10-times more sensitive to MTH-68/H than several other human cencer cell lines, such as HCT-116, DU-145 or NCI-H460 (FIG. 1A and Table I).

To confirm that the LNZTA3WT4 cell line is p53⁻ and p53⁺ in the presence and absence of tetracyclin, respectively, under the test conditions used for MTH-68/H infection, cells were subjected to Western-blot analysis after various treatments (see FIG. 4) using antibodies against components of the p53 signaling network. As FIG. 4A shows, LNZTA3WT4 cells did not express the p53 protein in the presence of tetracyclin, while in the absence of the antibiotic, p53 expression could be easily detected. Moreover, no significant p53 expression could be detected upon MTH-68/H infection in tetracyclin-treated cells.

Similar results were obtained for some of the components of the p53 regulatory network (FIG. 4A). PTEN, a lipid phosphatase responsible for the negative regulation of the Akt survival signaling pathway via the dephosphorylation of PIP₃, has recently been reported to be involved in the regulation of p53. Its activated form inhibits Akt-mediated Mdm2 phosphorylation, thereby increasing the stability of p53. On the other hand, PTEN is a p53-regulated tumor suppressor itself. Once p53 is stabilized, it increases transcription of the pten gene, elevating the PTEN protein level in the cell. Indeed, p53 and PTEN levels are tightly coregulated in LNZTA3WT4 glioblastoma cells: both are highly expressed in the absence of tetracyclin, but disappear from cells grown in tetracyclin-containing medium (FIG. 4A). In summary, these results indicate that expression of the exogenous p53 cDNA is regulated by tetracyclin under the test conditions used, and that the p53 protein synthesized in the absence of tetracyclin is functionally active. In the presence of tetracyclin, both p53 and PTEN expression is repressed and MTH-68/H infection hardly has any effect on the low level of these proteins.

At low PTEN activity (that can be caused by the low proteini level and/or phosphorylation of PTEN on Ser 388), the level of the second messenger PIP₃ increases (see above), Akt is activated by phosphorylation on Thr 308 and Ser 473, and, besides other target proteins, phosphorylates Mdm2 on Ser 166 that stimulates its nuclear translocation. Mdm2, being a ubiquitin ligase, induces ubiquitination and proteasomal degradation of the p53 protein, thereby leading to the stimulation of cell survival. The functional state of this anti-apoptotic pathway can, thus, easily be monitored by analyzing the phosphorylation levels of its key components, PTEN, Akt and Mdm2. Surprisingly, not only the level of PTEN is induced by tetracyclin withdrawal in LNZTA3WT4 cells, but its phosphorylation as well, which consequently leads to increased Akt (not shown) and Mdm2 phosphorylation (FIG. 4A). Induction of p53 and PTEN is pro-apoptotic, while the stimulation of PTEN/Akt/Mdm2 phosphorylation has a pro-survival effect. Presumably, the observed activation of this survival pathway is the result of the selection procedure during the isolation of the LNZTA3WT4 cell line: without this compensatory alteration, these cells would rapidly die in the absence of tetracyclin, due to the toxically elevated p53 protein level.

The effect of MTH-68/H on the phosphorylation of these proteins was also somewhat unexpected. MTH-68/H, while hardly affecting the level of PTEN, induced a transient phosphorylation of the protein that was accompanied by an increased phosphorylation of Akt and Mdm2 (FIGS. 4A and 4B). The extent of these phosphorylation events were slighter than those observed in the absence of tetracyclin. MTH-68/H-induced stimulation of the PTEN/Akt/Mdm2 pathway, on the other hand, is p53-independent: the p53 protein was hardly detectable during the 12-hour course of MTH-68/H infection. MTH-68/H, thus, efficiently induces apoptosis of p53-depleted glioblastoma cells, but, at the same time, stimulates survival mechanisms as well.

The Effect of MTH-68/H on Nuclear Enhancer Binding Activities

To further analyze the significance of the p53 pathway in MTH-68/H-induced cytotoxicity, electrophoretic mobility shift assays were performed with nuclear extracts of p53-positive and p53-negative cells, using an oligonucleotide probe with a p53-binding site. FIG. 5 shows the results of such tests with wtPC12 (p53+, panel A) and tetracyclin-treated LNZTA3WT4 cells (p53⁻, panel B). In wtPC12 cells (panel A) UV irradiation, as expected, strongly stimulated the enhancer-binding activity of p53 protein (sample 2). In contrast, MTH-68/H infection only slightly enhanced the DNA-binding of p53 (samples 3 to 8). Moderate increase in p53 activity upon MTH-68/H treatment was observed using HCT-116 colon carcinoma cells (p53+, not shown). In contrast, neither basal level (sample 1), nor UV-(sample 2) or MTH-68/H-induced (samples 3 to 8, panel B) p53 activity was observed in tetracyclin-treated LNZTA3WT4 glioblastoma cells.

Panels C and D in FIG. 5 present results of electromobility shift assays of the same nuclear extracts using an oligonucleotide with a c-Myc binding sequence. The results of this control experiment indicate that MTH-68/H stimulates c-Myc DNA-binding activity independently of the p53-status of the cell: both wtPC12 cells (panel C) and tetracyclin treated LNZTA3WT4 cells (panel D) respond similarly to virus infection. C-Myc is a transcription factor with both proliferation-stimulating and pro-apoptotic functions. The present results suggest that c-Myc is activated by MTH-68/H, and that it acts upstream or independently of p53.

While the present invention has been described in terms of specific embodiments thereof, it will be understood that no limitations are intended to the details of the disclosed methods other than as defined in the appended claims.

1. A method for treating p53-negative human tumor cells to induce apoptotic cell death thereof comprising the step of infecting the tumor cells with the Herefordshire strain of Newcastle disease virus.

2. A method, as claimed in claim 1, wherein the MOI is in the range of 100/1 to 1/200 cell/infective particle ratio.

3. A method, as claimed in claim 1, wherein the MOI is 1/10 cell/infective particle ratio.

4. A method, as claimed in claim 1, wherein the human tumor cells are selected from pancreas, glioblastoma, melanoma, colorectal, prostate, cervical and breast cancer cells.
